(19) United States
(12) Patent Application Publication
(10) Pub. No.: US 2026/0000336 A1
(43) Pub. Date: Jan. 1, 2026

(54) FIRST WEARABLE ELECTRONIC DEVICE FOR ACQUIRING SIGNAL INCLUDING NOISE CORRESPONDING TO MOVEMENT OF USER, ELECTRONIC DEVICE FOR ANALYZING SIGNAL, AND OPERATING METHOD OF ELECTRONIC DEVICE

(71) Applicant: SAMSUNG ELECTRONICS CO., LTD., Suwon-si (KR)

(72) Inventors: Hyunjin AHN, Suwon-si (KR); Seonho KIM, Suwon-si (KR); Yoosung KIM, Suwon-si (KR); Dasom PARK, Suwon-si (KR); Eunkyu OH, Suwon-si (KR)

(21) Appl. No.: 19/330,787

(22) Filed: Sep. 16, 2025

Related U.S. Application Data
(63) Continuation of application No. PCT/KR2024/002481, filed on Feb. 27, 2024.

(30) Foreign Application Priority Data
Apr. 18, 2023 (KR) 10-2023-0090378

(51) Int. Cl. A61B 5/369; A61B 5/00; A61B 5/0205; A61B 5/024

(52) U.S. Cl. CPC A61B 5/369; A61B 5/0205; A61B 5/6817; A61B 5/7207; A61B 5/02438

(57) ABSTRACT

A first wearable electronic device includes a first electrode, a communication circuitry, a processor, and a memory for storing instructions. The instructions cause the first wearable electronic device to: acquire, through the first electrode, a brainwave signal of a user and a first signal, which includes first noise caused by first movement of the user and second noise caused by second movement of the user; acquire, through the communication circuitry, from a second wearable electronic device, a second signal, which includes third noise caused by the second movement of the user measured by the second wearable electronic device; use the first signal and the second signal to acquire a third signal for confirming the first movement; and transmit, through the communication circuit, the third signal to the electronic device so that the electronic device executes a function corresponding to the first movement.

### CROSS-REFERENCE TO RELATED APPLICATIONS

This application is a continuation of International Application No. PCT/KR2024/002481 designating the United States, filed on Feb. 27, 2024, in the Korean Intellectual Property Receiving Office and claiming priority to Korean Patent Application No. 10-2023-0050908, filed on Apr. 18, 2023, and Korean Patent Application No. 10-2023-0090378, filed on Jul. 12, 2023, in the Korean Intellectual Property Office, the disclosure of each of which are herein incorporated by reference in their entireties.

### BACKGROUND

The disclosure relates to a first wearable electronic device for acquiring a signal including noise corresponding to a user's movement, an electronic device for analyzing the signal, and an operating method of the electronic device.

Biosignals include electroencephalography (EEG), electrocardiogram (ECG), ballistocardiogram (BCG), and photoplethysmogram (PPG). By analyzing such biosignals, an electronic device may obtain various types of information related to the state of a living organism.

Particularly, brainwaves are signals that reflect the state of the brain and are considered highly important among various biosignals. Brainwaves refer to electrical signals generated by brain activity. These brainwaves may be used in the diagnosis of neurological disorders such as epilepsy, stroke, and brain tumors. In recent years, brainwaves have also been used not only for disease diagnosis, but also for monitoring brain activity in subjects during perception and cognitive ability tests.

### SUMMARY

According to an embodiment, a first wearable electronic device may include a first electrode, a communication module, a processor, and memory connected electrically to the processor and configured to store instructions executable by the processor, wherein the instructions, when executed by the processor, cause the first wearable electronic device to perform operations.

According to an embodiment, the first wearable electronic device may acquire a first signal via the first electrode in a state where the first wearable electronic device is worn in one ear of a user, the first signal including a brainwave signal of the user, a first noise caused by a first movement of the user, and a second noise caused by a second movement of the user.

According to an embodiment, the first wearable electronic device may acquire, via the communication module, a second signal from a second wearable electronic device worn in an other ear of the user, the second signal being measured by the second wearable electronic device and including a third noise caused by the second movement of the user.

According to an embodiment, the first wearable electronic device may acquire, using the first signal and the second signal, a third signal for identifying the first movement.

According to an embodiment, the first wearable electronic device may transmit the third signal to an electronic device via the communication module such that the electronic device performs a function corresponding to the first movement.

According to an embodiment, a method for operating the first wearable electronic device may include acquiring a first signal via the first electrode in a state where the first wearable electronic device is worn in one ear of the user, the first signal including a brainwave signal of the user, a first noise caused by a first movement of the user, and a second noise caused by a second movement of the user.

According to an embodiment, the method for operating the first wearable electronic device may include acquiring, via the communication circuit of the first wearable electronic device, a second signal from a second wearable electronic device worn in another ear of the user, the second signal being measured by the second wearable electronic device and including a third noise caused by the second movement of the user.

According to an embodiment, the method for operating the first wearable electronic device may include acquiring, using the first signal and the second signal, a third signal for identifying the first movement.

According to an embodiment, the method for operating the first wearable electronic device may include transmitting the third signal to the electronic device via the communication circuit such that the electronic device performs a function corresponding to the first movement.

According to an embodiment, a non-transitory recording medium may include at least one instruction that enables an operation of acquiring a first signal via the first electrode in a state where the first wearable electronic device is worn in one ear of the user, the first signal including a brainwave signal of the user, a first noise caused by a first movement of the user, and a second noise caused by a second movement of the user.

According to an embodiment, the non-transitory recording medium may include at least one instruction that enables an operation of acquiring, via the communication module of the first wearable electronic device, a second signal from the second wearable electronic device worn in the other ear of the user, the second signal being measured by the second wearable electronic device and including a third noise caused by the second movement of the user.

According to an embodiment, the non-transitory recording medium may include at least one instruction that enables an operation of acquiring, using the first signal and the second signal, a third signal for identifying the first movement.

According to an embodiment, the non-transitory recording medium may include at least one instruction that enables an operation of transmitting the third signal to the electronic device via the communication module such that the electronic device performs a function corresponding to the first movement.

According to an embodiment, the electronic device may include a communication module, a processor, and memory connected electrically to the processor and configured to store instructions executable by the processor, wherein the instructions, when executed by the processor, cause the first wearable electronic device to perform operations.

According to an embodiment, the electronic device may acquire a third signal for identifying a first movement of a user, from the first wearable electronic device via the communication module.

According to an embodiment, the electronic device may acquire the third signal by using the first signal measured by the first wearable electronic device worn in one ear of the user and the second signal measured by the second wearable electronic device worn in the other ear of the user.

According to an embodiment, in the electronic device, the first signal may include a brainwave signal of the user, a first noise caused by a first movement of the user, and a second noise caused by a second movement of the user, and the second signal may include a third noise caused by the second movement.

According to an embodiment, the electronic device may perform a function of the electronic device corresponding to the first movement, based on the third signal.

According to an embodiment, a method for operating the electronic device may include acquiring a third signal for identifying a first movement of the user, from the first wearable electronic device via the communication module of the electronic device.

According to an embodiment, in the method for operating the electronic device, the third signal may be acquired using a first signal measured by the first wearable electronic device worn in one ear of the user and a second signal measured by the second wearable electronic device worn in the other ear of the user.

According to an embodiment, in the method for operating the electronic device, the first signal may include a brainwave signal of the user, a first noise caused by a first movement of the user, and a second noise caused by a second movement of the user, and the second signal may include a third noise caused by the second movement.

According to an embodiment, the method for operating the electronic device may include performing a function of the electronic device, corresponding to the first movement, based on the third signal.

According to an embodiment, a non-transitory recording medium may include at least one instruction that enables an operation of acquiring a third signal for identifying a first movement of the user, from the first wearable electronic device via the communication module of the electronic device.

According to an embodiment, in the non-transitory recording medium, the third signal may be acquired using a first signal measured by the first wearable electronic device worn in one ear of the user and a second signal measured by the second wearable electronic device worn in the other ear of the user.

According to an embodiment, in the non-transitory recording medium, the first signal may include a brainwave signal of the user, a first noise caused by a first movement of the user, and a second noise caused by a second movement of the user, and the second signal may include a third noise caused by the second movement.

According to an embodiment, the non-transitory recording medium may include at least one instruction that enables an operation of performing a function of the electronic device corresponding to the first movement, based on the third signal.

### BRIEF DESCRIPTION OF THE DRAWINGS

FIG. 1 is a block diagram of an electronic device in a network environment according to various embodiments.

FIG. 2 illustrates a system for analyzing noise corresponding to a user's movement, according to an embodiment.

FIG. 3A illustrates a first wearable electronic device and a second wearable electronic device according to an embodiment.

FIG. 3B illustrates one ear of a user that may be used to wear a first wearable electronic device according to an embodiment.

FIG. 4 is a schematic diagram for a system for analyzing noise corresponding to a user's movement, according to an embodiment.

FIG. 5 is a flowchart illustrating an operation in which an electronic device according to an embodiment executes a function of the electronic device, based on signals acquired by a first wearable electronic device and a second wearable electronic device.

FIG. 6 is a flowchart illustrating an operation in which an electronic device according to an embodiment executes a function of the electronic device, based on a third signal.

FIG. 7 is a flowchart illustrating an operation in which an electronic device according to an embodiment executes a function of the electronic device, based on a third signal.

FIG. 8 is a flowchart illustrating an operation in which an electronic device according to an embodiment executes a function of the electronic device, based on a third signal.

FIG. 9 is a flowchart illustrating an operation in which an electronic device according to an embodiment executes a function of the electronic device, based on a third signal.

FIG. 10 is a flowchart illustrating an operation in which an electronic device according to an embodiment executes a function of the electronic device, based on a third signal.

FIG. 11 is a flowchart illustrating an operation in which an electronic device according to an embodiment executes a function of the electronic device, based on a third signal.

FIG. 12A illustrates a signal including a brainwave signal and noise caused by an eye blink, according to an embodiment.

FIG. 12B illustrates a signal including a brainwave signal and noise caused by a jaw movement, according to an embodiment.

FIG. 13 illustrates an operation in which an electronic device according to an embodiment configures a designated movement.

FIG. 14A illustrates an operation in which an electronic device according to an embodiment acquires information related to a movement, based on a third signal.

FIG. 14B illustrates an operation in which an electronic device according to an embodiment provides information related to an eye blink.

FIG. 14C illustrates an operation in which an electronic device according to an embodiment provides guide information for eye stretching.

FIG. 15 illustrates an operation in which an electronic device according to an embodiment unlocks the electronic device, based on a third signal.

### DETAILED DESCRIPTION

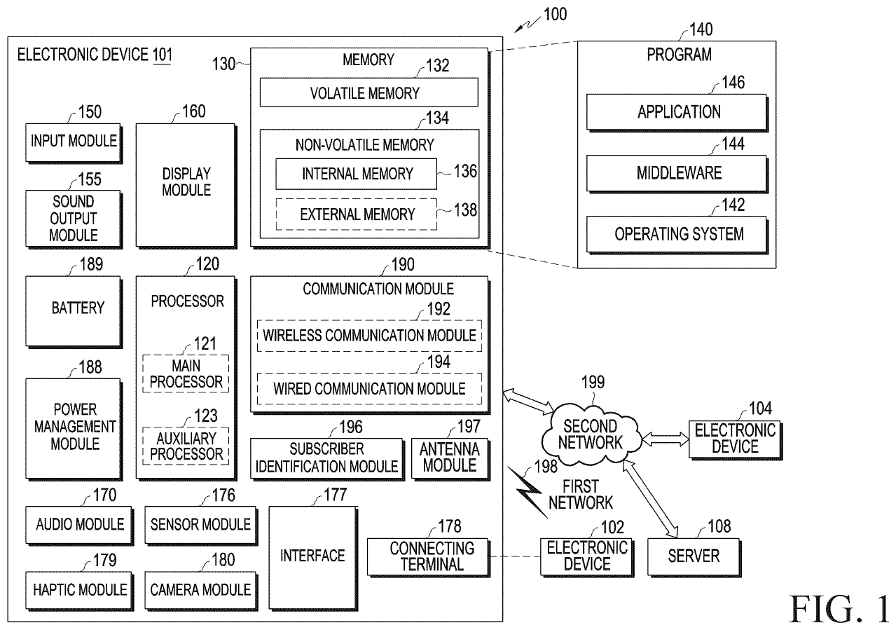

FIG. 1 is a block diagram illustrating an electronic device 101 in a network environment 100 according to various embodiments. Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted Boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of Ims or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

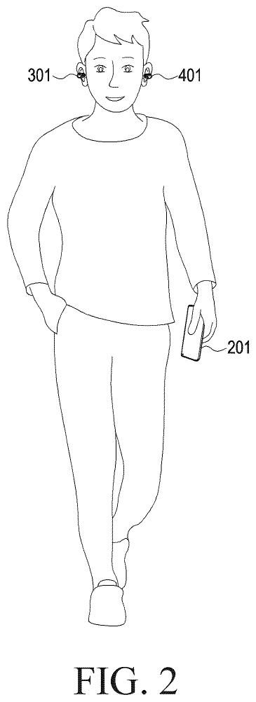

FIG. 2 illustrates a system for analyzing noise corresponding to a user's movement, according to an embodiment.

Referring to FIG. 2, according to an embodiment, a first wearable electronic device 301 may be worn in one ear of a user. A second wearable electronic device 401 may be worn in the other ear of the user. For example, the first wearable electronic device 301 and the second wearable electronic device 401 may be implemented as a pair of wireless earphones. For example, the electronic device 201 may be implemented as a smartphone or a tablet PC.

According to an embodiment, the electronic device 201 may identify a user's movement by analyzing a signal (e.g., a signal including a brainwave and noise) received from the first wearable electronic device 301 while the first wearable electronic device 301 is worn in one ear of the user. According to an embodiment, the user's movement may include a user's eye blink, a user's jaw movement, a user's heartbeat, and/or the like. For example, the electronic device 201 may analyze the signal received from the first wearable electronic device 301 by using an artificial intelligence (AI) model. For example, the AI model may be prestored in the electronic device 201.

According to an embodiment, the electronic device 201 may identify a user's movement by analyzing a signal (e.g., a signal including a brainwave and noise) received from the second wearable electronic device 401 while the second wearable electronic device 401 is worn in the other ear of the user. However, for convenience of explanation, the disclosure will focus on a method in which the electronic device 201 identifies a user's movement by analyzing a signal (e.g., a signal including a brainwave and noise) received from the first wearable electronic device 301. The technical features of the disclosure may be equally applied to a method in which the electronic device 201 identifies a user's movement by analyzing a signal (e.g., a signal including a brainwave and noise) received from the second wearable electronic device 401.

According to an embodiment, the electronic device 201 may identify whether the user's movement identified based on the signal (e.g., the signal including a brainwave and noise) received from the first wearable electronic device 301 corresponds to a movement predesignated by the electronic device 201 or by the user. When it is identified that the identified user's movement corresponds to a designated movement, the electronic device 201 may execute a function of the electronic device 201 corresponding to the designated movement. For example, the function of the electronic device 201 may include a function of controlling a music application of the electronic device 201, a function of locking or unlocking the electronic device 201, a function of sensing or outputting information on a user's sleep state, a function of sensing or outputting information on a user's exercise state, a function of outputting information on the heart rate, a function of sensing or outputting information on a user's meal, and/or the like.

Existing electronic devices analyzed a signal including user's brainwave and noise from the first wearable electronic device, thereby identifying the user's brainwave. However, existing electronic devices only identified the brainwaves and did not analyze the noise separated from the signal. In other words, existing electronic devices did not analyze the noise included in the signal including a brainwave and noise, nor did the existing electronic devices utilize the analyzed noise as meaningful data.

The electronic device 201 according to an embodiment may identify noise from a signal (e.g., a third signal) including a brainwave and noise, received from the first wearable electronic device 301 or the second wearable electronic device 401 and utilize the identified noise as meaningful data. For example, the electronic device 201 may identify a user's movement indicated by noise and perform a function of the electronic device 201, based on the identified movement.

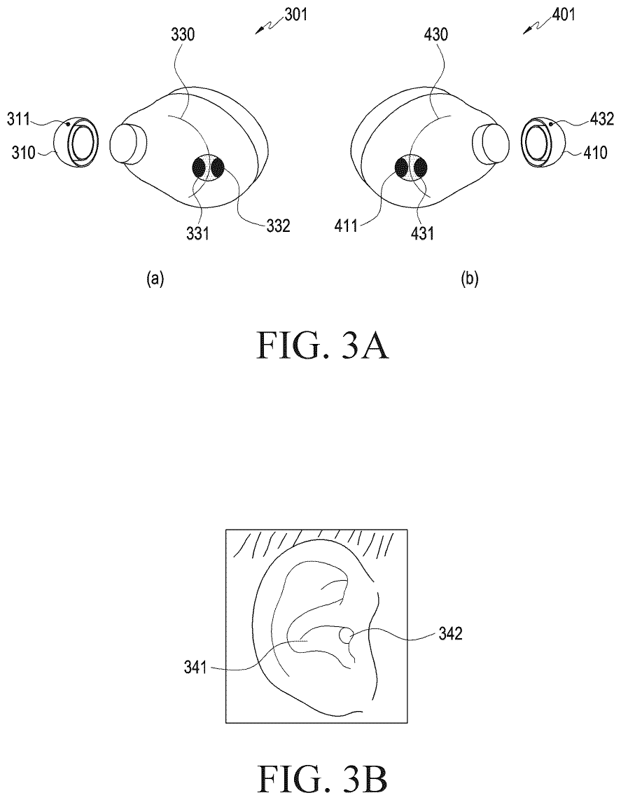

FIG. 3A illustrates a first wearable electronic device and a second wearable electronic device according to an embodiment.

Referring to (a) of FIG. 3A, according to an embodiment, the first wearable electronic device 301 (e.g., the first wearable electronic device 301 of FIG. 2) may include an ear tip 310 and a main body (or a body part) 330 coupled to the ear tip 310.

According to an embodiment, the first wearable electronic device 301 may include a first electrode 311, a first ground electrode 331, and a third electrode 332.

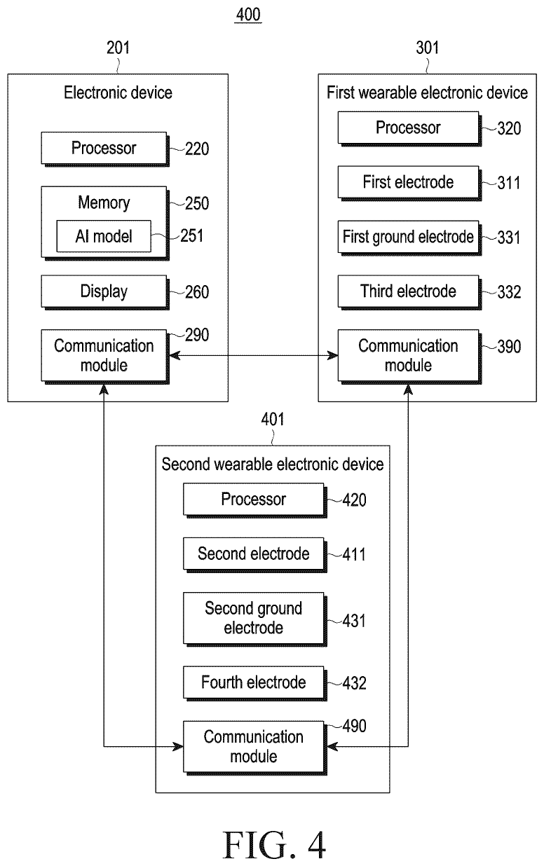

According to an embodiment, a processor 320 of the first wearable electronic device 301 (e.g., the processor 320 of FIG. 4) may be placed in the main body 330. According to an embodiment, the processor 320 may be connected to the first electrode 311, the first ground electrode 331, and the third electrode 332. However, this is an example, and the processor 320 may be placed in the ear tip 310.

According to an embodiment, the first electrode 311 may be disposed on the ear tip 310. According to an embodiment, the first electrode 311 may include at least one electrode (e.g., a conductive electrode) exposed to the outside so as to be in contact with the user's ear. According to the implementation, the first electrode 311 may be implemented with a plurality of electrodes. However, the number or shape of the electrodes included in the first electrode 311 may be variously modified in a manner understood by those skilled in the art. According to an embodiment, the first electrode 311 may be implemented with silver (Ag). However, this is an example, and the material of the first electrode 311 may not be limited thereto. According to an embodiment, the first ground electrode 331 may be disposed on the main body 330. According to an embodiment, the first ground electrode 331 may be a ground for generating a potential difference. According to an embodiment, the third electrode 332 may be placed on the main body 330. For example, the first ground electrode 331 and the third electrode 332 may be exposed to the outside of the main body 330 so as to be in contact with the user's ear.

Referring to (b) of FIG. 3A, according to an embodiment, the second wearable electronic device 401 (e.g., the second wearable electronic device 401 of FIG. 2) may include an ear tip 410 and a main body (or body part) 430. According to an embodiment, the main body 430 may be detachable from the ear tip 410.

According to an embodiment, the second wearable electronic device 401 may include a second electrode 411, a second ground electrode 431, and a fourth electrode 432.

According to an embodiment, a processor 420 of the second wearable electronic device 401 (e.g., the processor 420 of FIG. 4) may be disposed in the main body 430. According to an embodiment, the processor 420 may be connected to the second electrode 411, the second ground electrode 431, and the fourth electrode 432. However, this is only an example, and the processor 420 may also be placed in the ear tip 410.

According to an embodiment, the second electrode 411 may be disposed on the main body 430. According to an embodiment, the fourth electrode 432 may be disposed on the ear tip 410. According to an embodiment, the fourth electrode 432 may include at least one electrode (e.g., a conductive electrode) exposed to the outside so as to be in contact with the user's ear. According to the implementation, the fourth electrode 432 may be implemented with a plurality of electrodes. However, the number or shape of the electrodes included in the fourth electrode 432 may be variously modified in a manner understood by those skilled in the art. According to an embodiment, the fourth electrode 432 may be implemented with silver (Ag). However, this is an example, and the material of the fourth electrode 432 may not be limited thereto. According to an embodiment, the second ground electrode 431 may be disposed on the main body 430. According to an embodiment, the second ground electrode 431 may be a ground for generating a potential difference. For example, the second ground electrode 431 and the second electrode 411 may be exposed to the outside of the main body 430 so as to be in contact with the user's ear.

FIG. 3B illustrates one ear of a user that can be used to wear a first wearable electronic device according to an embodiment.

Referring to FIG. 3B, according to an embodiment, the first wearable electronic device 301 may be worn in one ear of a user. According to an embodiment, the first electrode 311 included in the first wearable electronic device 301 may be in contact with the hole 342 of one ear of the user. According to an embodiment, the first ground electrode 331 included in the first wearable electronic device 301 may be in contact with the concha 341 of one ear of the user. According to an embodiment, the third electrode 332 included in the first wearable electronic device 301 may be in contact with the concha 341 of one ear of the user.

According to an embodiment, the second wearable electronic device 401 may be worn in the other ear of the user. According to an embodiment, the fourth electrode 432 included in the second wearable electronic device 401 may be in contact with the hole (not shown) of the other ear of the user. According to an embodiment, the second ground electrode 431 included in the second wearable electronic device 401 may be in contact with the concha (not shown) of the other ear of the user. According to an embodiment, the second electrode 411 included in the second wearable electronic device 401 may be in contact with the concha (not shown) of the other ear of the user.

According to an embodiment, the first wearable electronic device 301 may acquire a first signal via the first electrode 311 while being worn in one ear of the user, the first signal including a brainwave signal, a first noise caused by a first movement, and a second noise caused by a second movement. According to an embodiment, the second wearable electronic device 401 may acquire a second signal including a third noise caused by the second movement, through the second electrode 411 while being worn in the other ear of the user. According to an embodiment, the second signal may be used as a reference signal for removing a portion corresponding to the second noise from the first signal. According to an embodiment, the first movement may include a user's eye blink, jaw movement, eye (eyeball) up/down/left/right movement, or heartbeat. According to an embodiment, the second movement may include a user's head movement, a user's arm movement, or a user's foot movement. However, this is an example, and embodiments of the disclosure may not be limited to the examples of the first movement and the second movement described above.

According to an embodiment, the first wearable electronic device 301 may acquire, by using the first signal and the second signal, a third signal for identifying the first movement. According to an embodiment, the first wearable electronic device 301 may transmit the third signal to the electronic device 201. According to an embodiment, the electronic device 201 may analyze the third signal and identify the first movement, based on the analysis result of the third signal. According to an embodiment, the electronic device 201 may execute a function corresponding to the first movement.

Alternatively, according to an embodiment, the second wearable electronic device 401 may acquire a fourth signal including a brainwave signal, a fifth noise caused by the first movement, and a sixth noise caused by the second movement, through the fourth electrode 432 while being worn in the other ear of the user. According to an embodiment, the first wearable electronic device 301 may acquire a fifth signal including a seventh noise caused by the second movement, through the third electrode 332. According to an embodiment, the fifth signal may be used as a reference signal for removing a portion corresponding to the sixth noise caused by the second movement.

According to an embodiment, the second wearable electronic device 401 may acquire a sixth signal for identifying the first movement by using the fourth signal and the fifth signal. According to an embodiment, the second wearable electronic device 401 may transmit the sixth signal to the electronic device 201. According to an embodiment, the electronic device 201 may analyze the sixth signal and identify the first movement, based on the analysis result of the sixth signal. According to an embodiment, the electronic device 201 may execute a function corresponding to the first movement.

Hereinafter, for convenience of explanation, the electronic device 201 is described as executing a function corresponding to a first movement, based on the third signal acquired using the first electrode 311 included in the first wearable electronic device 301, the first ground electrode 331 included in the first wearable electronic device 301, the second electrode 411 included in the second wearable electronic device 401, and the second ground electrode 431 included in the second wearable electronic device 401. However, the technical idea of the disclosure is not limited thereto.

FIG. 4 is a schematic diagram of a system for analyzing a user's brainwave signal according to an embodiment.

Referring to FIG. 4, a system 400 for analyzing a user's brainwave signal may include an electronic device 201, a first wearable electronic device 301, and a second wearable electronic device 401.

According to an embodiment, the first wearable electronic device 301 and the second wearable electronic device 401 may be implemented as wearable electronic devices that are wearable in the user's ears. For example, the first wearable electronic device 301 and the second wearable electronic device 401 may be implemented as true wireless stereo (TWS) devices (hereinafter, wireless earphones). According to an embodiment, the first wearable electronic device 301 may be worn in one ear of the user, and the second wearable electronic device 401 may be worn in the other ear of the user. According to an embodiment, the electronic device 201 may be implemented as a smartphone or a tablet PC. However, this is only an example, and the first wearable electronic device 301, the second wearable electronic device 401, and the electronic device 201 of the disclosure may be implemented as various types of devices.

According to an embodiment, the first wearable electronic device 301 may include memory (not shown), a processor 320, a first electrode 311, a first ground electrode 331, a third electrode 332, and a communication module 390. According to an embodiment, the memory (not shown) may be implemented in a manner identical or similar to the memory 130 of FIG. 1.

According to an embodiment, the second wearable electronic device 401 may include memory (not shown), a processor 420, a second electrode 411, a second ground electrode 431, a fourth electrode 432, and a communication module 490. According to an embodiment, the memory (not shown) may be implemented in a manner identical or similar to the memory 130 of FIG. 1.

According to an embodiment, the processor 320 may control overall operations of the first wearable electronic device 301. According to an embodiment, the processor 420 may control overall operations of the second wearable electronic device 401.

According to an embodiment, the processor 320 of the first wearable electronic device 301 may acquire, via the first electrode 311, a first signal including a user's brainwave signal, a first noise caused by a user's first movement, and a second noise caused by a user's second movement. The first noise may refer to noise caused by the first movement, and the second noise may refer to noise caused by the second movement. According to an embodiment, the first signal may be measured by the first electrode 311 that is in contact with the hole 342 of one ear wearing the first wearable electronic device 301. According to an embodiment, the first movement may include a user's eye blink, jaw movement, eye (eyeball) up/down/left/right movement, or heartbeat. According to an embodiment, the second movement may include a user's head movement, a user's arm movement, a user's torso movement, or a user's foot movement. However, this is an example, and embodiments of the disclosure may not be limited to the examples of the first movement and the second movement described above.

According to an embodiment, the processor 420 of the second wearable electronic device 401 may acquire a second signal including a third noise caused by a user's second movement, via the second electrode 411. According to an embodiment, the second signal may be measured by the second electrode 411 that is in contact with the concha of the other ear wearing the second wearable electronic device 401. According to an embodiment, the amplitude of the second noise and the amplitude of the third noise may be identical or similar. According to an embodiment, the second signal may further include a fourth noise caused by a user's first movement. The third noise may refer to noise signal caused by the second movement, and the fourth noise may refer to noise signal caused by the first movement. According to an embodiment, the amplitude of the first noise and the amplitude of the second noise may be different. For example, the amplitude of the first noise may be greater than the amplitude of the fourth noise. In an embodiment, the second signal may be used as a reference signal for removing, from the first signal, the second noise caused by the second movement. In an embodiment, the processor 420 may transmit the second signal to the first wearable electronic device 301 via the communication module 490.

According to an embodiment, the processor 320 may acquire a second signal from the second wearable electronic device 401 via the communication module 390. According to an embodiment, the processor 320 may acquire, by using the first signal and the second signal, a third signal for identifying the first movement.

According to an embodiment, the processor 320 may remove a portion corresponding to the second noise from the first signal by using the second signal. According to an embodiment, the processor 320 may also remove a portion corresponding to a portion of the first noise and a portion corresponding to the second noise from the first signal by using the second signal. For example, when the amplitude of the fourth noise included in the second signal is smaller than the amplitude of the first noise included in the first signal, the first wearable electronic device 301 may remove a portion corresponding to a portion of the first noise from the first signal.

According to an embodiment, the processor 320 may separate, by using a fast Fourier transform (FFT), a signal remaining after removing a portion corresponding to the second noise from the first signal into a portion corresponding to the brainwave signal and a portion corresponding to the first noise. For example, in the signal remaining after removing a portion corresponding to the second noise from the first signal, the third signal may refer to a signal corresponding to a portion corresponding to the first noise.

According to an embodiment, the processor 320 may transmit the third signal to the electronic device 201 via the communication module 390. In addition, the processor 320 may also transmit, to the electronic device 201, a signal corresponding to a portion corresponding to the brainwave signal from the signal remaining after removing a portion corresponding to the second noise from the first signal.

According to the implementation, the processor 320 may also transmit, to the electronic device 201, a signal remaining after removing a portion corresponding to the second noise from the first signal without separating a portion corresponding to the first noise by using a fast Fourier transform. At this time, the electronic device 201 may separate a portion corresponding to the first noise from the signal by using a fast Fourier transform. Accordingly, the electronic device 201 may acquire a third signal.

According to the implementation, the processor 320 may transmit the first signal and the second signal to the electronic device 201 without removing the portion corresponding to the second noise from the first signal. At this time, the electronic device 201 may remove the portion corresponding to the second noise from the first signal by using the second signal, and may separate the portion corresponding to the first noise from the signal by using a fast Fourier transform. Accordingly, the electronic device 201 may acquire a third signal.

According to an embodiment, the electronic device 201 may include a processor 220, memory 250, a display 260, and a communication module 290. According to an embodiment, the processor 220 may control overall operations of the electronic device 201. For example, the processor 220 may be implemented in a manner identical to or similar to the processor 120 of FIG. 1.

According to an embodiment, the processor 220 may acquire a third signal from the first wearable electronic device 301 via the communication module 290.

According to an embodiment, the memory 250 may store an artificial intelligence (AI) model 251. The AI model 251 may be implemented in software. The AI model 251 may refer to a model that is trained to output, from at least one signal including at least one noise caused by at least one movement and/or a brainwave signal, information on the at least one movement. According to an embodiment, the AI model may output information on at least one movement as an analysis result, based on the shape of the waveform of at least one signal and/or the amplitude of the waveform of the at least one signal. For example, the waveform of a signal representing a user's conscious movement and the waveform of a signal representing a user's unconscious movement may be different. For example, the amplitude of the waveform of a signal representing a conscious movement may be greater than the amplitude of the waveform of a signal representing an unconscious movement. The user's movement for performing a function of the electronic device 201, described in the disclosure may refer to a user's conscious movement.

According to an embodiment, waveforms of signals representing noises caused by different movements may be different from each other. For example, the waveform shape of a signal including noise caused by a heartbeat may be different from the waveform shape of a signal including noise caused by an eye blink. In addition, even for noise caused by the same movement, the waveform of a signal represented by the noise may be different depending on the number of times the movement occurs. For example, the waveform of a signal corresponding to noise caused by one eye blink may be different from the waveform of a signal corresponding to noise caused by two eye blinks. For example, the amplitude of the waveform of a signal including noise caused by two eye blinks may be larger than the amplitude of the waveform of a signal including noise caused by one eye blink.

According to an embodiment, the processor 220 may analyze the third signal by using the prestored AI model 251. For example, the processor 220 may input the third signal into the AI model 251 and identify the first movement indicated by the third signal. According to an embodiment, the processor 220 may identify whether the first movement corresponds to a predesignated movement. According to an embodiment, when it is identified that the first movement corresponds to the predesignated movement, the processor 220 may execute a predesignated function related to the predesignated movement.

According to an embodiment, the predesignated movement may include a movement related to a user's eye blink, jaw movement, or heartbeat. For example, when the user's heart rate obtained for a preconfigured period of time is greater than or equal to the preconfigured number, the processor 220 may identify that the movement corresponds to the predesignated movement. When the number of the user's jaw movements obtained for a preconfigured period of time is equal to the preconfigured number of times, the processor 220 may identify that the movement corresponds to the predesignated movement. When the number of the user's eye blinks obtained for a preconfigured period of time is equal to the preconfigured number of times, the processor 220 may identify that the movement corresponds to the predesignated movement. When the number of the user's left-right eye movements obtained for a preconfigured period of time is equal to the preconfigured number of times, the processor 220 may identify that the movement corresponds to the predesignated movement. When the user has kept his/her eyes open without blinking for a preconfigured period of time or when the user's eyes are closed for a preconfigured period of time, the processor 220 may identify that the movement corresponds to a predesignated movement.

According to an embodiment, the number of the user's eye blinks may refer to the number of the user's conscious eye blinks. According to an embodiment, the waveform shape of the noise obtained when the user blinks his/her eyes unconsciously may be different from the waveform shape of the noise obtained when the user blinks his/her eyes consciously. For example, the amplitude of the noise obtained when the user blinks his/her eyes unconsciously may be smaller than the amplitude of the noise obtained when the user blinks his/her eyes consciously. For example, the amplitude of the noise may refer to the amplitude of the noise waveform. For example, the processor 220 may not include the number of the user's unconscious eye blinks in the number of eye blinks. In addition, the processor 220 may not include other types of user's unconscious movements in the number of movements used to perform a function of the electronic device 201.

According to an embodiment, the predesignated functions may include a function of controlling a music application of the electronic device 201, a function of locking or unlocking the electronic device 201, a function of sensing or outputting information on a user's sleep state, a function of sensing or outputting information on a user's exercise state, a function of outputting information on the heart rate, or a function of sensing or outputting information on a user's meal, among others.

According to an embodiment, the processor 220 may unlock the electronic device 201 when it is identified, based on the third signal, that the user blinks his/her eyes once within a preconfigured period of time. The processor 220 may lock the electronic device 201 when it is identified, based on the third signal, that the user blinks his/her eyes two times within a preconfigured period of time.

According to an embodiment, the processor 220 may play music (or execute a music application) on the electronic device 201 when it is identified, based on the third signal, that the user blinks his/her eyes once within a preconfigured period of time. The processor 220 may stop playing the music when it is identified, based on the third signal, that the user blinks his/her eyes two times within a preconfigured period of time.

According to an embodiment, the processor 220 may output notification information that prompts the user to blink his/her eyes when it is identified, based on the third signal, that the user has kept his/her eyes open without blinking for a preconfigured period of time.

According to an embodiment, the processor 220 may output notification information indicating a user's sleep state when it is identified, based on the third signal, that the user's eyes are closed for a preconfigured period of time.

According to an embodiment, when the number of the user's heartbeats for a preconfigured period of time is identified to be greater than the preconfigured number of times, based on the third signal, the processor 220 may output notification information indicating an abnormality in the number of the heartbeats. For example, the preconfigured number of times may refer to the number of times the user's heart is identified as abnormal.

According to an embodiment, when it is identified, based on the third signal, that the user's jaw moves at a regular interval for a preconfigured period of time, the processor 220 may identify that the user is eating and identify the mealtime of the user. The processor 220 may store the mealtime of the user in the memory 250.

According to an embodiment, when it is identified, based on the third signal, that the user's jaw moves at a regular interval for a preconfigured period of time and that the user's eyes are closed, the processor 220 may identify that the user is grinding his/her teeth while sleeping. When it is identified that the user is grinding his/her teeth while sleeping, the processor 220 may output notification information indicating a teeth-grinding state.

According to an embodiment, when it is identified, based on the third signal, that the user's jaw moves at a regular interval for a preconfigured period of time and a voice signal outside of the first wearable electronic device 301 or outside of the second wearable electronic device 401, which is obtained by a microphone (not shown) included in the first wearable electronic device 301 or a microphone (not shown) included in the second wearable electronic device 401, is obtained via the communication module 290, the processor 220 may identify that the user is in a speaking state. When it is identified that the user is in a speaking state, the processor 220 may stop the execution of the music application or reduce the volume of the music application.

According to an embodiment, the processor 220 may also analyze the brainwave signal via the AI model 251. In addition, the processor 220 may provide the analysis result of the brainwave signal to the user via the display (not shown) or speaker (not shown) included in the electronic device 201.

According to an embodiment, when the amplitude of the brainwave signal is identified to be smaller than a preconfigured amplitude, the processor 220 may output notification information indicating an abnormal wearing state of the first wearable electronic device 301. The amplitude of the brainwave signal may refer to the amplitude of the waveform of the brainwave signal. For example, when it is identified that the shape of the waveform of the brainwave signal is not identical or similar to the shape of the preconfigured waveform, the processor 220 may output notification information indicating an abnormal wearing state of the first wearable electronic device 301. For example, when the amplitude of the waveform of the brainwave signal is identified to be smaller than a preconfigured amplitude, the processor 220 may also output notification information for ear cleaning of one ear wearing the first wearable electronic device 301. The preconfigured amplitude may refer to the amplitude at which the first wearable electronic device 301 is identified to be in an abnormal wearing state.

According to an embodiment, when it is identified, based on the third signal, that the user's eyes are closed for a preconfigured period of time and the brainwave signal (e.g., alpha wave) included in the third signal is identified as a signal acquired when the user is in a sleeping state, the processor 220 may output notification information indicating the user's sleeping state.

According to an embodiment, when it is identified that the user's jaw moves at a regular interval for a preconfigured period of time and the brainwave signal included in the third signal is identified as a brainwave signal acquired when the user is in a good mood, the processor 220 may display, on the display 260, a popup window that prompts the user to input the type of food currently being eaten, the name of the food, or the name of the restaurant. The processor 220 may also provide the type of food eaten by other users, the name of the food, or the name of the restaurant.

According to an embodiment, when the brainwave signal included in the third signal is identified as a brainwave signal acquired when the user is drinking excessively, the processor 220 may output notification information indicating that the user appears to be under the influence of a substance.

According to an embodiment, when the brainwave signal included in the third signal is identified as a brainwave signal acquired when the user is in a concentrated state, the processor 220 may identify and store the user's concentration time.

However, this is an example, and the predesignated movements and predesignated functions of the embodiments of the disclosure may not be limited to the above examples.

The operations of the electronic device 201 described in the drawings below may be performed by the processor 220. However, for convenience of explanation, the operations performed by the processor 220 will be described as being performed by the electronic device 201. The operations of the first wearable electronic device 301 described in the drawings below may be performed by the processor 320. However, for convenience of explanation, the operations performed by the processor 320 will be described as being performed by the first wearable electronic device 301. The operations of the second wearable electronic device 401 described in the drawings below may be performed by the processor 420. However, for convenience of explanation, the operations performed by the processor 420 will be described as being performed by the second wearable electronic device 401.

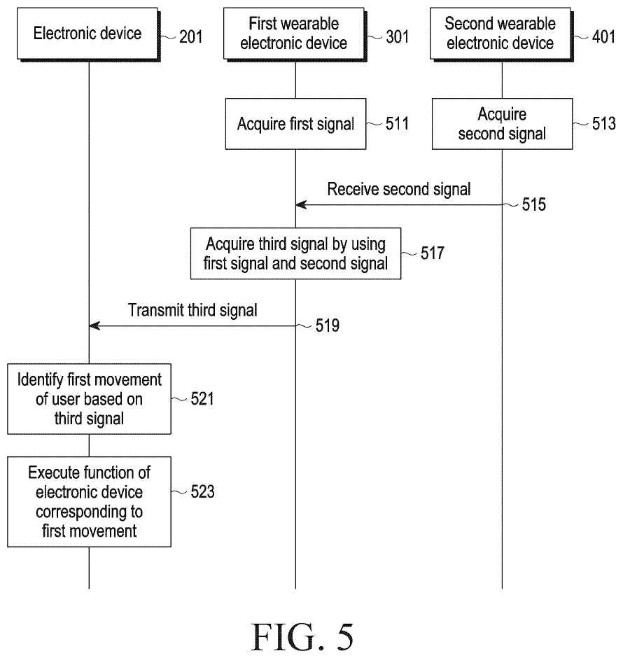

FIG. 5 is a flowchart illustrating an operation in which an electronic device according to an embodiment executes a function of the electronic device, based on signals acquired by a first wearable electronic device and a second wearable electronic device.

Referring to FIG. 5, according to an embodiment, in operation 511, the first wearable electronic device 301 (e.g., the first wearable electronic device 301 of FIG. 4) may acquire a first signal. According to an embodiment, the first wearable electronic device 301 may measure the first signal via the first electrode 311 (e.g., the first electrode 311 of FIG. 4) while being worn in one ear of a user. According to an embodiment, the first electrode 311 may be in contact with a portion corresponding to the hole 342 of one ear of the user. According to an embodiment, the first signal may include a user's brainwave signal, a first noise caused by a user's first movement, and a second noise caused by a user's second movement. In an embodiment, the first movement may include a user's eye blink, a user's jaw movement, or a user's heartbeat. In an embodiment, the second movement may include a user's head movement, a user's arm movement, a user's torso movement, or a user's foot movement.

According to an embodiment, in operation 513, the second wearable electronic device 401 (e.g., the second wearable electronic device 401 of FIG. 4) may acquire a second signal. According to an embodiment, the second wearable electronic device 401 may measure the second signal via the second electrode 411 (e.g., the second electrode 411 of FIG. 4) while being worn in the other ear of the user. According to an embodiment, the second signal may include a third noise caused by the second movement. According to an embodiment, the second electrode 411 may be in contact with the concha of the other ear of the user. According to an embodiment, the shape and/or amplitude of the waveform of the second noise may be identical or similar to the shape and/or amplitude of the waveform of the third noise. According to an embodiment, the second signal may further include a fourth noise caused by the first movement. The amplitude of the first noise may be different from the amplitude of the fourth noise. For example, the amplitude of the first noise may be greater than the amplitude of the fourth noise. The amplitude of the noise may refer to an amplitude of the noise waveform.

According to an embodiment, in operation 515, the first wearable electronic device 301 may receive the second signal from the second wearable electronic device 401 via the communication module 390 (e.g., the communication module 390 of FIG. 4).

According to an embodiment, in operation 517, the first wearable electronic device 301 may acquire a third signal by using the first signal and the second signal. For example, the third signal may be a signal for allowing the electronic device (e.g., the electronic device 201 of FIG. 3B) to identify the user's first movement. According to an embodiment, the second signal may be used as a reference signal for removing, from the first signal, the second noise caused by the second movement.

According to an embodiment, the first wearable electronic device 301 may remove a portion corresponding to the second noise from the first signal by using the second signal when acquiring or generating the third signal.

According to an embodiment, the first wearable electronic device 301 may also remove a portion corresponding to the first noise and a portion corresponding to the second noise from the first signal by using the second signal when acquiring or generating the third signal. For example, when the amplitude of the fourth noise included in the second signal is smaller than the amplitude of the first noise included in the first signal, the first wearable electronic device 301 may also remove a portion of the first noise from the first signal.

According to an embodiment, the first wearable electronic device 301 may separate, by using a fast Fourier transform (FFT), a signal remaining after removing the second noise from the first signal into a portion corresponding to the brainwave signal and a portion corresponding to the first noise. The first wearable electronic device 301 may obtain, as the third signal, a signal corresponding to the portion corresponding to the first noise among the portion corresponding to the brainwave signal and the portion corresponding to the first noise. For example, in the signal remaining after removing the second noise from the first signal, the third signal may refer to a signal corresponding to the portion corresponding to the first noise. In this case, the first wearable electronic device 301 may acquire a third signal by amplifying the portion corresponding to the first noise by a designated amplitude.

According to an embodiment, in operation 519, the first wearable electronic device 301 may transmit the third signal to the electronic device 201 (e.g., the electronic device 201 of FIG. 4) via the communication module 390.

According to an embodiment, in operation 521, the electronic device 201 may identify the user's first movement, based on the third signal. According to an embodiment, the electronic device 201 may analyze the third signal by using an AI model 251 (e.g., the AI model 251 of FIG. 4) prestored in the memory 250 (e.g., the memory 250). For example, the electronic device 201 may input the third signal into the AI model 251 to identify the first movement indicated by the third signal. In addition, the electronic device 201 may identify whether the identified first movement corresponds to a movement predesignated by the electronic device 201 or the user.

According to an embodiment, in operation 523, when it is identified that the identified first movement corresponds to a movement predesignated by the electronic device 201 or the user, the electronic device 201 may execute a function of the electronic device 201 corresponding to the first movement. According to an embodiment, the function of the electronic device 201 corresponding to the first movement may include a function of controlling a music application of the electronic device 201, a function of locking or unlocking the electronic device 201, a function of sensing or outputting information on a user's sleep state, a function of sensing or outputting information on a user's exercise state, a function of outputting information on the heart rate, or a function of sensing or outputting information on a user's meal.

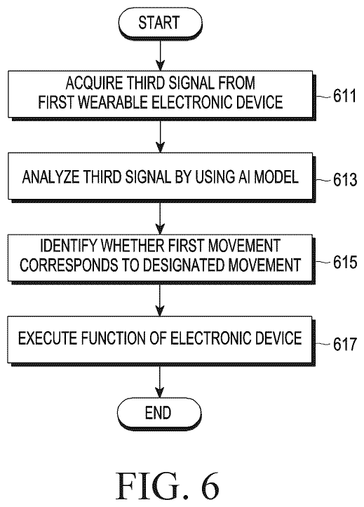

FIG. 6 is a flowchart for illustrating an operation in which an electronic device according to an embodiment executes a function of the electronic device, based on a third signal.

Referring to FIG. 6, according to an embodiment, in operation 611, the electronic device 201 (e.g., the electronic device 201 of FIG. 4) may acquire a third signal from the first wearable electronic device 301 (e.g., the first wearable electronic device 301 of FIG. 4). According to an embodiment, the third signal may refer to a signal corresponding to a portion corresponding to the first noise, the signal being derived from a signal remaining after removing a portion corresponding to a second noise from a first signal.

According to an embodiment, in operation 613, the electronic device 201 may analyze the third signal by using the AI model 251 (e.g., the AI model 251 of FIG. 4) prestored in the memory 250 (e.g., the memory 250 of FIG. 4). The AI model 251 may refer to a model that outputs, from at least one signal including at least one noise caused by at least one movement and/or a brainwave signal, information on the at least one movement. According to an embodiment, the AI model may output information on at least one movement as an analysis result, based on the shape of the waveform of at least one signal and/or the amplitude of the waveform of the at least one signal. For example, the electronic device 201 may input the third signal into the AI model 251 and identify information on the first movement corresponding to the third signal as an analysis result by the AI model 251. For example, information on the first movement may include information on the number of the user's heartbeats for a preconfigured period of time, information on the user's jaw movement for a preconfigured period of time, information on the number of the user's eye blinks for a preconfigured period of time, information on the user's eye (e.g., eyeball) up/down/left/right movements for a preconfigured period of time, or information on the user's eyes being closed for a preconfigured period of time. For example, information on the number of the user's eye blinks may refer to information on the number of the user's conscious eye blinks.

According to an embodiment, in operation 615, the electronic device 201 may identify whether the first movement corresponds to a predesignated movement.

According to an embodiment, when the number of user's heartbeats for a preconfigured period of time is greater than the preconfigured number of times, the electronic device 201 may identify that the movement corresponds to a predesignated movement.

According to an embodiment, when the user's jaw moves at a regular interval or the number of the user's jaw movements for a preconfigured period of time is greater than the preconfigured number of times, the electronic device 201 may identify that the movement corresponds to a predesignated movement.

According to an embodiment, when the number of the user's eye blinks for a preconfigured period of time is equal to the preconfigured number of times, the electronic device 201 may identify that the movement corresponds to a predesignated movement.

According to an embodiment, when the user's eyes move from left to right (or from right to left) a preconfigured number of times within a preconfigured period of time, the electronic device 201 may identify that the movement corresponds to a predesignated movement.

According to an embodiment, when the user's eyes move from bottom to top (or from top to bottom) a preconfigured number of times within a preconfigured period of time, the electronic device 201 may identify that the movement corresponds to a predesignated movement.

According to an embodiment, when the user's eyes are closed for a preconfigured period of time, the electronic device 201 may identify that the movement corresponds to a predesignated movement. However, this is an example, and embodiments of the disclosure may not be limited to the above-described movements.

According to an embodiment, in operation 617, when it is identified that the first movement corresponds to the designated movement, the electronic device 201 may execute a function of the electronic device 201 corresponding to the designated movement. According to an embodiment, the predesignated function may include a function of controlling a music application of the electronic device 201, a function of locking or unlocking the electronic device 201, a function of sensing or outputting information on a user's sleep state, a function of sensing or outputting information on a user's exercise state, a function of outputting information on the heart rate, or a function of sensing or outputting information on a user's meal. However, this is an example, and embodiments of the disclosure may not be limited to the above-described functions.

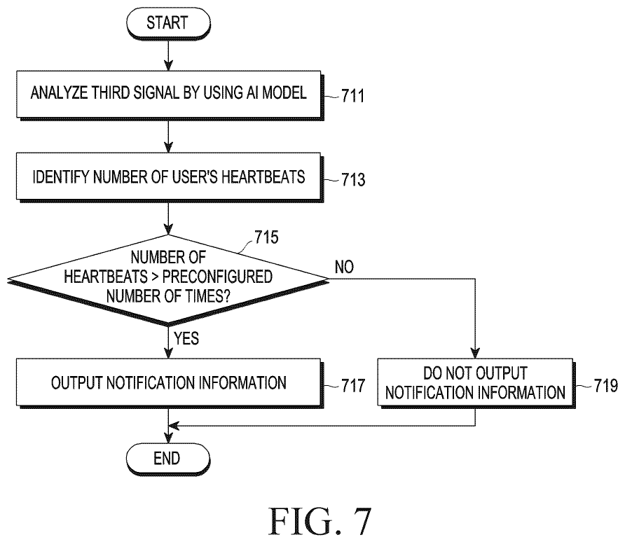

FIG. 7 is a flowchart illustrating an operation in which an electronic device according to an embodiment executes a function of the electronic device, based on a third signal.

Referring to FIG. 7, according to an embodiment, in operation 711, the electronic device 201 (e.g., the electronic device 201 of FIG. 4) may analyze the third signal by using the AI model 251 (e.g., the AI model 251 of FIG. 4) prestored in the memory 250 (e.g., the memory 250 of FIG. 4). For example, the electronic device 201 may input the third signal into the AI model 251 and identify information on the first movement corresponding to the third signal, based on the analysis result by the AI model 251. For example, the electronic device 201 may identify that the first movement represents the user's heartbeat, based on the third signal input into the AI model 251.

According to an embodiment, in operation 713, the electronic device 201 may identify the number of the user's heartbeats, based on the third signal analyzed via the AI model 251. According to an embodiment, the electronic device 201 may identify the number of the user's heartbeats, based on the third signal input into the AI model 251.

According to an embodiment, in operation 715, the electronic device 201 may identify whether the number of the user's heartbeats is greater than the preconfigured number of times. For example, the preconfigured number of times may refer to the number of times the user's heart is identified as abnormal.

According to an embodiment, in operation 717, when the heart rate is identified to be greater than the preconfigured number of times (operation 715-Yes), the electronic device 201 may output notification information indicating an abnormality in the number of the user's heartbeats. According to an embodiment, the electronic device 201 may output the notification information onto the display 260 (e.g., the display 260 of FIG. 4). According to one embodiment, the electronic device 201 may transmit a control signal to the first wearable electronic device 301 and/or the second wearable electronic device 401 to cause the first wearable electronic device 301 and/or the second wearable electronic device 401 to output notification information.

According to an embodiment, in operation 719, when the heart rate is identified not to be greater than the preconfigured number of times (operation 715-No), the electronic device 201 may not output notification information indicating an abnormality in the number of the user's heartbeats.

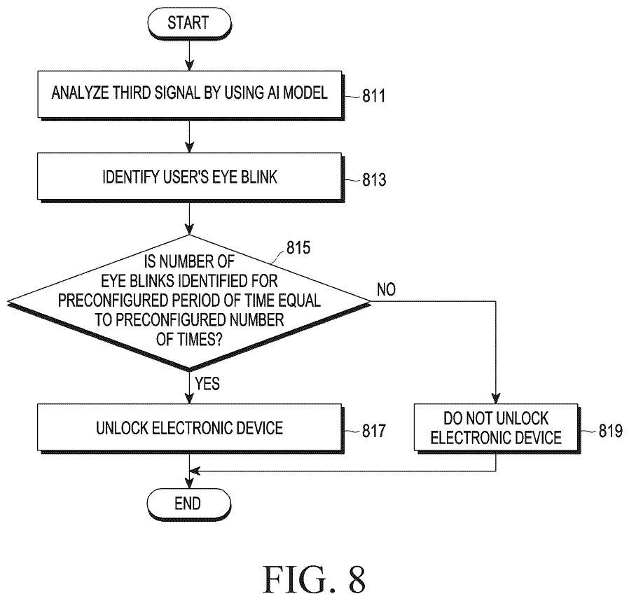

FIG. 8 is a flowchart illustrating an operation in which an electronic device according to an embodiment executes a function of the electronic device, based on a third signal.

Referring to FIG. 8, according to an embodiment, in operation 811, the electronic device 201 (e.g., the electronic device 201 of FIG. 4) may analyze the third signal by using the AI model 251 (e.g., the AI model 251 of FIG. 4). For example, the electronic device 201 may input the third signal into the AI model 251 and may identify information on the first movement corresponding to the third signal, based on the analysis result by the AI model 251.

According to an embodiment, in operation 813, the electronic device 201 may identify that the first movement indicates the user's eye blink, based on the analysis of the third signal via the AI model 251. According to an embodiment, the eye blink may refer to a user's conscious eye blinking movement.

According to an embodiment, in operation 815, the electronic device 201 may identify whether the number of the user's eye blinks is equal to the preconfigured number of times. According to an embodiment, the electronic device 201 may identify the number of the user's eye blinks, based on the third signal input into the AI model 251. For example, the AI model 251 may identify the number of the user's eye blinks, based on the shape and/or the amplitude of the waveform of the third signal. For example, the more the user blinks his/her eyes, the greater the amplitude of the third signal may be.

According to an embodiment, in operation 817, when the number of eye blinks is identified to be equal to the preconfigured number of times (e.g., once) (operation 815-Yes), the electronic device 201 may be unlocked.

According to an embodiment, in operation 819, when the number of eye blinks is identified not to be equal to the preconfigured number of times (operation 815-No), the electronic device 201 may not be unlocked.

According to an embodiment, when the number of eye blinks identified to be equal to the preconfigured number of times (e.g., once), the electronic device 201 may execute a music application of the electronic device 201 to play music. According to an embodiment, when the number of eye blinks is identified to be equal to the preconfigured number of times (e.g., two times), the electronic device 201 may stop playing music of the electronic device 201. According to an embodiment, the electronic device 201 may play the next track in the currently playing music when the number of eye blinks is identified to be equal to the preconfigured number of times (e.g., three times).

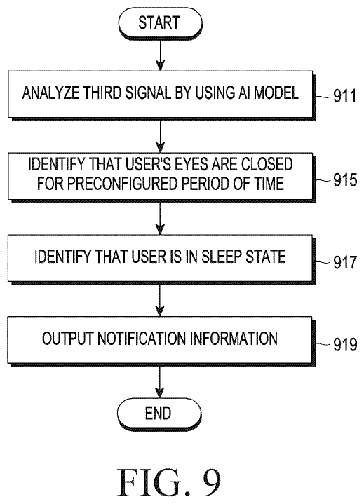

FIG. 9 is a flowchart illustrating an operation in which an electronic device according to an embodiment executes a function of the electronic device, based on a third signal.

Referring to FIG. 9, according to an embodiment, in operation 911, the electronic device 201 (e.g., the electronic device 201 of FIG. 4) may analyze the third signal by using the AI model 251 (e.g., the AI model 251 of FIG. 4) prestored in the memory 250 (e.g., the memory 250 of FIG. 4). According to an embodiment, the electronic device 201 may input the third signal into the AI model 251.

According to an embodiment, in operation 915, the electronic device 201 may identify that the user's eyes are closed for a preconfigured period of time, based on the analysis of the third signal via the AI model 251.

According to an embodiment, in operation 917, the electronic device 201 may identify that the user is in a sleeping state, based on the identification that the user's eyes are closed.

According to an embodiment, in operation 919, the electronic device 201 may output notification information indicating the user's sleep state. For example, the notification information indicating the sleep state may be notification information that prompts the user to interrupt the sleep state. According to an embodiment, the electronic device 201 may output the notification information through a speaker (not shown) included in the electronic device 201. According to an embodiment, the electronic device 201 may also transmit a control signal to the first wearable electronic device 301 and/or the second wearable electronic device 401 to output the notification information through a speaker (not shown) included in the first wearable electronic device 301 (e.g., the first wearable electronic device 301 of FIG. 4) and/or a speaker (not shown) included in the second wearable electronic device 401 (e.g., the second wearable electronic device 401 of FIG. 4).

According to an embodiment, the first wearable electronic device 301 may also transmit, to the electronic device 201, a signal corresponding to a portion corresponding to the brainwave signal, from the signal remaining after removing the second noise from the first signal. According to an embodiment, the electronic device 201 may analyze the brainwave signal. For example, the electronic device 201 may analyze the brainwave signal by using the AI model 251. For example, when it is identified, based on the third signal, that the user's eyes are closed for a preconfigured period time and the brainwave signal is identified as a signal obtained when the user is in a sleeping state (e.g., alpha wave), the electronic device 201 may output notification information indicating the user's sleeping state.

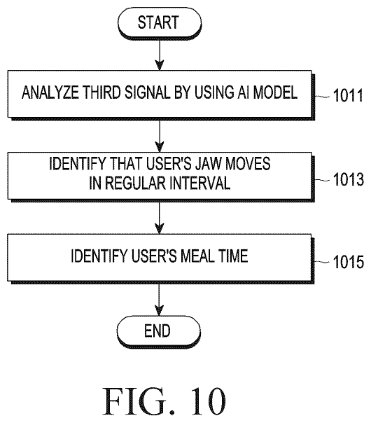

FIG. 10 is a flowchart illustrating an operation in which an electronic device according to an embodiment executes a function of the electronic device, based on a third signal.

Referring to FIG. 10, according to an embodiment, in operation 1011, the electronic device 201 (e.g., the electronic device 201 of FIG. 4) may analyze the third signal by using the AI model 251 (e.g., the AI model 251 of FIG. 4) prestored in the memory 250 (e.g., the memory 250 of FIG. 4).

According to an embodiment, in operation 1013, the electronic device 201 may identify that the user's jaw moves at a regular interval, based on the analysis of the third signal via the AI model 251. For example, the electronic device 201 may input the third signal into the AI model 251 to identify that the first movement represents a jaw movement. The electronic device 201 may identify via the AI model 251 that the user's jaw moves at regular intervals.

According to an embodiment, in operation 1015, the electronic device 201 may identify the user's mealtime, based on the identification that the user's jaw moves at a regular interval. According to an embodiment, when it is identified that the user's jaw moves at a regular interval, the electronic device 201 may identify that the user is eating. According to an embodiment, when it is identified that the user is eating, the electronic device 201 may identify the mealtime of the user. For example, the user's mealtime may refer to a period from a time point at which the user's jaw is identified as moving to a timepoint at which the user's jaw is identified as not moving.

According to an embodiment, the electronic device 201 may also analyze a brainwave signal. For example, the electronic device 201 may analyze a brainwave signal by using the AI model 251. According to an embodiment, the electronic device 201 may also analyze a brainwave signal. For example, the electronic device 201 may also analyze a brainwave signal through the AI model 251. According to an embodiment, when it is identified that the user's jaw moves at a regular interval for a preconfigured period of time and the brainwave signal is identified as a brainwave signal acquired when the user is in a good mood, the electronic device 201 may display, on the display 260 (e.g., the display 260 of FIG. 4), a popup window that prompts the user to input the type of food currently being eaten, the name of the food, or the name of the restaurant. The electronic device 201 may also provide the type of food eaten by other users, the name of the food, or the name of the restaurant.

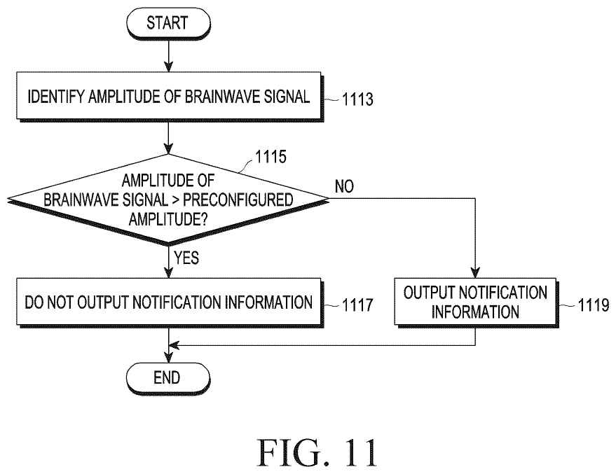

FIG. 11 is a flowchart illustrating an operation in which an electronic device according to an embodiment executes a function of the electronic device, based on a third signal.

Referring to FIG. 11, according to an embodiment, in operation 1113, the electronic device 201 (e.g., the electronic device 201 of FIG. 4) may identify the amplitude of the brainwave signal. According to an embodiment, the first wearable electronic device 301 (e.g., the first wearable electronic device 301 of FIG. 4) may also transmit, to the electronic device 201, a signal corresponding to a portion corresponding to the brainwave signal, from the signal remaining after removing the second noise from the first signal.

According to an embodiment, in operation 1115, the electronic device 201 may identify whether the amplitude of the brainwave signal is greater than a preconfigured amplitude. The preconfigured amplitude may refer to the amplitude at which whether the first wearable electronic device 301 is identified to be in an abnormal wearing state.

According to an embodiment, in operation 1117, when the amplitude of the brainwave signal is identified to be greater than a preconfigured amplitude (operation 1115-Yes), the electronic device 201 may not output notification information.

According to an embodiment, in operation 1119, when it is identified that the amplitude of the brainwave signal is not greater than the preconfigured amplitude (operation 1115-No), the electronic device 201 may output notification information. According to an embodiment, the electronic device 201 may output notification information on ear cleaning of one ear wearing the first wearable electronic device 301. According to an embodiment, the electronic device 201 may output notification information that guides the wearing state of the first wearable electronic device 301. According to an embodiment, the electronic device 201 may also transmit a control signal to the first wearable electronic device 301 and/or the second wearable electronic device 401 to cause the first wearable electronic device 301 and/or the second wearable electronic device 401 to output the notification information.

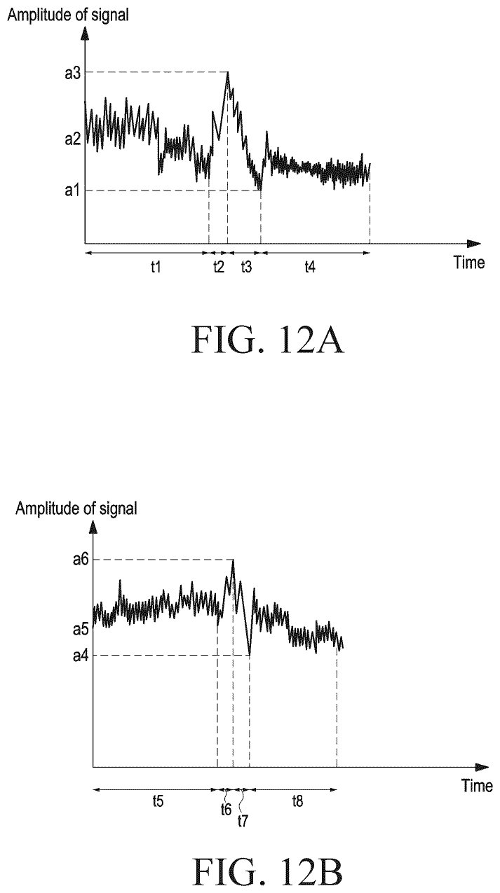

FIG. 12A illustrates a signal including a brainwave signal and noise caused by an eye blink, according to an embodiment.

Referring to FIG. 12A, according to an embodiment, the first wearable electronic device 301 (e.g., the first wearable electronic device 301 of FIG. 4) may acquire a first signal. The first signal may include a user's brainwave signal, a first noise caused by a user's first movement, and a second noise caused by a user's second movement. According to an embodiment, the second wearable electronic device 401 may acquire a second signal. The second signal may include a third noise caused by the user's second movement. According to an embodiment, the first wearable electronic device 301 may remove, by using the second signal, a portion corresponding to the second noise caused by the user's second movement from the first signal. According to an embodiment, the first movement may include a user's eye blinking movement. For example, the first movement may refer to a user's conscious eye blinking movement. In an embodiment, the second movement may include a user's head movement, a user's arm movement, or a user's foot movement.

According to an embodiment, the first wearable electronic device 301 may acquire a signal remaining after removing, from the first signal, by using the second signal, a portion corresponding to the second noise caused by the second movement. According to an embodiment, the signal illustrated in the graph of FIG. 12A may be a signal remaining after removing, from the first signal, by using the second signal, a portion corresponding to the second noise caused by the second movement.

According to an embodiment, the signal acquired during the first time interval (t1) may include a user's brainwave signal and noise caused by a user's unconscious eye blinking movement.

According to an embodiment, the amplitude of the signal acquired during the second time interval (t2) after the first time interval (t1) may increase from a2 to a3. According to an embodiment, the amplitude of the signal acquired during the second time interval (t2) may increase by noise caused by a user's conscious eye-closing movement.

In an embodiment, the amplitude of the signal acquired during a third time interval (t3) after the second time interval (t2) may decrease from a3 to a1. In an embodiment, the third time interval (t3) may be a period during which the user continues to consciously keep his/her eyes closed after the second time interval (t2).

According to an embodiment, the amplitude of the signal acquired during the fourth time interval (t4) after the third time interval (t3) may increase from a1 to a2 then be maintained at approximately a2. According to an embodiment, the amplitude of the third signal may increase from a1 to a2 by noise caused by a user's conscious eye-opening movement. According to an embodiment, the amplitude of the third signal may converge to approximately a2 by noise caused by a user's unconscious eye blinking movement. However, this is an example, and the waveform and amplitude of the signal of the disclosure may not be limited thereto.

FIG. 12B illustrates a signal including a brainwave signal and noise caused by a jaw movement, according to an embodiment.

Referring to FIG. 12B, according to an embodiment, the signal illustrated in the graph of FIG. 12B may be a signal remaining after removing, from the first signal, by using the second signal, a portion corresponding to the second noise caused by the second movement.

According to an embodiment, the signal acquired during the fifth time interval (t5) may include a user's brainwave signal.

According to an embodiment, the amplitude of the signal acquired during the sixth time interval (t6) after the fifth time interval (t5) may increase from a5 to a6, and the amplitude of the signal acquired during the seventh time interval (t7) after the sixth time interval (t6) may decrease from a6 to a4. According to an embodiment, the amplitude of the signal acquired during the sixth time interval (t6) may be changed by noise caused by a user's jaw movement.

According to an embodiment, the eighth time interval (t8) after the seventh time interval (t7) may be a period during which the user does not move his/her jaw. However, this is an example, and the waveform and amplitude of the signal of the disclosure may not be limited thereto.

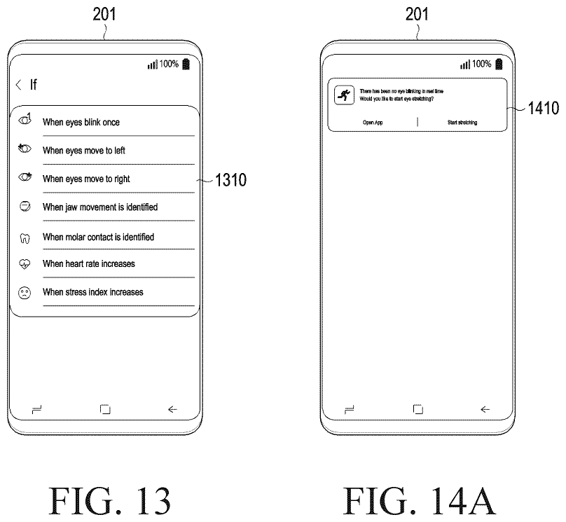

FIG. 13 illustrates an operation in which an electronic device according to an embodiment configures a designated movement.

Referring to FIG. 13, according to an embodiment, the electronic device 201 (e.g., the electronic device 201 of FIG. 4) may display, on the display 260 (e.g., the display 260 of FIG. 4), a screen 1310 that allows at least one movement to be designated for at least one function of the electronic device 201.

According to an embodiment, the at least one function may include a function of controlling a music application of the electronic device 201, a function of locking or unlocking the electronic device 201, a function of sensing or outputting information on a user's sleep state, or a function of sensing or outputting information on a user's exercise state. However, this is an example, and embodiments of the disclosure may not be limited thereto.

In an embodiment, the at least one movement may include blinking the eyes a preconfigured number of times (e.g., once, twice), not blinking the eyes for a preconfigured period of time, moving the eyes to the right, moving the eyes to the left, moving the jaw, clenching the molars, an increase in the heart rate, or an increase in stress index. However, this is an example and the embodiments of the disclosure may not be limited thereto.

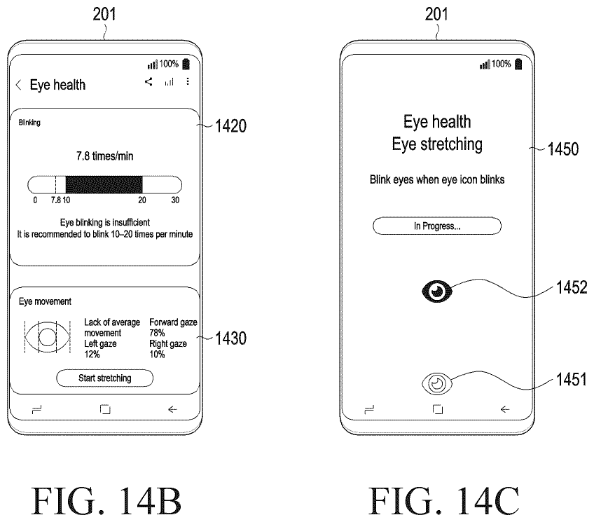

FIG. 14A illustrates an operation in which an electronic device according to an embodiment acquires information related to a movement, based on a third signal.

Referring to FIG. 14A, according to an embodiment, the electronic device 201 (e.g., the electronic device 201 of FIG. 4) may identify that the first movement included in the third signal indicates that the user has not blinks his/her eyes for a predetermined period of time.

According to an embodiment, when it is identified that the user has not blinks his/her eyes for a predetermined period of time, the electronic device 201 may display, on the display 260 (e.g., the display (260) of FIG. 4), a screen 1410 including information related to the user's movement.

According to an embodiment, the information related to the user's movement may include information indicating that the user has not blinks his/her eyes for a predetermined period of time (e.g., “There has been no eye blinking in real time. Would you like to start eye stretching?”), a first object (e.g., “Open App”) configured to display a screen through which information related to the user's eye blinking may be identified, and a second object (e.g., “Start Stretching”) configured to display a screen containing guidance information for eye stretching.

FIG. 14B illustrates an operation in which an electronic device according to an embodiment provides information related to an eye blink.

Referring to FIG. 14B, according to an embodiment, when an input to the first object (e.g., “Open App”) configured to display a screen through which information related to the user's eye blinking may be identified is identified, the electronic device 201 (e.g., the electronic device 201 of FIG. 4) may display, on the display 260 (e.g., the display 260 of FIG. 4), a screen 1420 including information on the number of eye blinks and a screen 1430 including information on an eye movement.

According to an embodiment, the information on the number of eye blinks may include the number of the user's eye blinks per minute (e.g., 7.8 times/min) and a recommended number of eye blinks per minute (e.g., 10 to 20 times/min).

According to an embodiment, the information on an eye movement may include information on the ratio of the user's forward gaze, left gaze, and right gaze. According to an embodiment, the screen 1430 including information on an eye movement may include a third object (e.g., “Start Stretching”) configured to provide guidance information for eye stretching.

FIG. 14C illustrates an operation in which an electronic device according to an embodiment provides guide information for eye stretching.

Referring to FIG. 14C, according to an embodiment, the electronic device 201 (e.g., the electronic device 201 of FIG. 4) may display, on the display 260 (e.g., the display 260 of FIG. 4), a screen 1450 including guide information for eye stretching.

According to an embodiment, when an input to the second object (e.g., “Start Stretching”) configured to display a screen containing guidance information for eye stretching is identified, the electronic device 201 (e.g., the electronic device 201 of FIG. 4) may display the screen 1450 including the guide information for eye stretching.

According to an embodiment, a screen 1450 including guide information for eye stretching may include a first icon 1451 and a second icon 1452.

According to an embodiment, the first icon 1451 may be displayed at a first position, and the display position of the first icon 1451 may remain unchanged. For example, the first position may refer to the bottom portion of the screen 1450 that includes the guide information for eye stretching. According to an embodiment, the display position of the second icon 1452 may change over time from a second position to the first position. For example, the second position may refer to a location relatively above the first position.

According to an embodiment, the screen 1450 including the guide information for eye stretching may include information prompting the user to blink his/her eyes when the display of the second icon 1452 overlaps the display location of the first icon 1451 (e.g., “Blink your eyes when the eye icons overlap”).

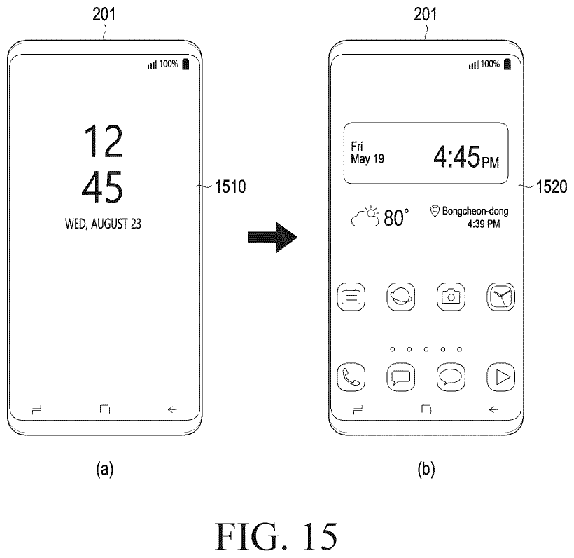

FIG. 15 illustrates an operation in which an electronic device according to an embodiment unlocks the electronic device, based on a third signal.

Referring to (a) of FIG. 15, according to an embodiment, the electronic device 201 (e.g., the electronic device 201 of FIG. 4) may display a lock screen 1510 of the electronic device 201. According to an embodiment, the electronic device 201 may not unlock the electronic device 201 until a predetermined movement is identified.

Referring to (b) of FIG. 15, according to an embodiment, the electronic device 201 may acquire a third signal. According to an embodiment, the electronic device 201 may analyze the third signal by using the AI model 251 (e.g., the AI model 251 of FIG. 4). For example, the electronic device 201 may input the third signal into the AI model 251 to identify that the user's first movement indicates eye blink. In an embodiment, the electronic device 201 may identify that the user blinks his/her eyes once within a preconfigured period of time.

According to an embodiment, the electronic device 201 may unlock the electronic device 201, based on the user's eyes having blinked once.

According to an embodiment, a first wearable electronic device 301 may include a first electrode 311, a communication module 390, a processor 320, and memory storing instructions.

According to an embodiment, the first wearable electronic device 301 may acquire a first signal via the first electrode 311 while the first wearable electronic device 301 is worn in one ear of the user, the first signal including a brainwave signal of a user, a first noise caused by a first movement of the user, and a second noise caused by a second movement of the user.

According to an embodiment, the first wearable electronic device 301 may acquire a second signal from a second wearable electronic device 302 via the communication module 390, the second signal including a third noise caused by the second movement of the user and being measured by the second wearable electronic device 302 worn in the other ear of the user.

According to an embodiment, the first wearable electronic device 301 may acquire, by using the first signal and the second signal, a third signal for identifying the first movement.

According to an embodiment, the first wearable electronic device 301 may transmit the third signal to an electronic device 201 via the communication module such that the electronic device executes a function corresponding to the first movement.

According to an embodiment, in the first wearable electronic device 301, the second signal may be used as a reference signal for removing, from the first signal, the second noise caused by the second movement.

According to an embodiment, in the first wearable electronic device 301, the first signal may be measured by the first electrode 311 of the first wearable electronic device 301, the first electrode being in contact with the hole of one ear of the user.

According to an embodiment, in the first wearable electronic device 301, the second signal may be measured by a second electrode 411 of the second wearable electronic device 302, the second electrode being in contact with the concha of the other ear of the user.

According to an embodiment, the first wearable electronic device 301 may generate the third signal by removing a portion corresponding to the second noise from the first signal by using the second signal.

According to an embodiment, in the first wearable electronic device 301, the first movement may include an eye blink of the user, a jaw movement of the user, or a heartbeat of the user.

According to an embodiment, in the first wearable electronic device 301, the second movement may include a head movement of the user, an arm movement of the user, or a foot movement of the user.

According to an embodiment, the first wearable electronic device 301 may further include a ground electrode configured to be in contact with a concha portion of one ear of the user.

According to an embodiment, a method for operating a first wearable electronic device may include acquiring a first signal via the first electrode 311 while the first wearable electronic device 301 is worn in one ear of the user, the first signal including a brainwave signal of the user, a first noise caused by a first movement of the user, and a second noise caused by a second movement of the user.

According to an embodiment, the method for operating the first wearable electronic device may include acquiring a second signal from the second wearable electronic device 302 via the communication module 390 of the first wearable electronic device 301, the second signal including a third noise caused by the second movement of the user and being measured by the second wearable electronic device 302 worn in the other ear of the user.

According to an embodiment, the method for operating the first wearable electronic device 301 may include acquiring, by using the first signal and the second signal, a third signal for identifying the first movement.

According to an embodiment, the method for operating the first wearable electronic device 301 may include transmitting the third signal to the electronic device 201 via the communication module such that the electronic device executes a function corresponding to the first movement.

According to an embodiment, in the method for operating the first wearable electronic device 301, the second signal may be used as a reference signal for removing, from the first signal, the second noise caused by the second movement.

According to an embodiment, in the method for operating the first wearable electronic device 301, the first signal may be measured by the first electrode of the first wearable electronic device 301, the first electrode being in contact with the hole of one ear of the user.

According to an embodiment, in the method for operating the first wearable electronic device 301, the second signal may be measured by the second electrode of the second wearable electronic device 302, the second electrode being in contact with the concha of the other ear of the user.

According to an embodiment, in the method for operating the first wearable electronic device 301, the third signal may be generated by removing a portion corresponding to the second noise from the first signal by using the second signal.

According to an embodiment, in the method for operating the first wearable electronic device 301, the first movement may include an eye blink of the user, a jaw movement of the user, or a heartbeat of the user.

According to an embodiment, in the method for operating the first wearable electronic device 301, the second movement may include a head movement of the user, an arm movement of the user, or a foot movement of the user.

According to an embodiment, in the method for operating the first wearable electronic device 301, the first wearable electronic device 301 may further include a ground electrode configured to be in contact with a concha portion of one ear of the user.

According to an embodiment, a non-transitory recording medium may include at least one instruction that enables an operation of acquiring a first signal via the first electrode 311 while the first wearable electronic device 301 is worn in one ear of the user, the first signal including a brainwave signal of the user, a first noise caused by a first movement of the user, and a second noise caused by a second movement of the user.

According to an embodiment, the non-transitory recording medium may include at least one instruction that enables an operation of acquiring a second signal from the second wearable electronic device 302 via the communication circuit of the first wearable electronic device, the second signal including a third noise caused by the second movement of the user and being measured by the second wearable electronic device 302 worn in the other ear of the user.

According to an embodiment, the non-transitory recording medium may include at least one instruction that enables an operation of acquiring, by using the first signal and the second signal, a third signal for identifying the first movement.

According to an embodiment, the non-transitory recording medium may include at least one instruction that enables an operation of transmitting the third signal to the electronic device 201 via the communication circuit such that the electronic device executes a function corresponding to the first movement.

According to an embodiment, the electronic device 201 may include a communication module 290, a processor 220, and memory 250 storing instructions.

According to an embodiment, the electronic device 201 may acquire a third signal for identifying the first movement of the user, from a first wearable electronic device 301 via the communication module 290.

According to an embodiment, in the electronic device 201, the third signal may be acquired using the first signal measured by the first wearable electronic device 301 worn in one ear of the user and the second signal measured by a second wearable electronic device 302 worn in the other ear of the user.

According to an embodiment, in the electronic device 201, the first signal may include a brainwave signal of the user, a first noise caused by a first movement of the user, and a second noise caused by a second movement of the user, and the second signal may include a third noise caused by the second movement.

According to an embodiment, the electronic device 201 may execute a function of the electronic device corresponding to the first movement, based on the third signal.

According to an embodiment, in the electronic device 201, the first movement may include an eye blink of the user, a jaw movement of the user, or a heartbeat of the user.

According to an embodiment, in the electronic device 201, the first signal may be measured by the first electrode 311 of the first wearable electronic device 301, the first electrode being in contact with the hole of one ear of the user.

According to an embodiment, in the electronic device 201, the second signal may be measured by the second electrode 411 of the second wearable electronic device 401, the second electrode being in contact with the concha of the other ear of the user.

According to an embodiment, in the electronic device 201, the third signal may be generated by removing a portion corresponding to the second noise from the first signal by using the second signal.

According to an embodiment, in the electronic device 201, the function of the electronic device 201 may include a function of controlling a music application of the electronic device 201, a function of locking or unlocking the electronic device 201, a function of sensing or outputting information on a sleep state of the user, and a function of sensing or outputting information on an exercise state of the user.

According to an embodiment, the electronic device 201 may input the third signal into an AI model prestored in the electronic device 201 to determine whether to execute the function of the electronic device 201.

According to an embodiment, the electronic device 201 may input the third signal into the AI model prestored in the electronic device 201 to determine whether the first movement indicated by the third signal corresponds to a predesignated movement related to the function.

According to an embodiment, the electronic device 201 may execute the function of the electronic device 201 when it is identified that the first movement corresponds to the predesignated movement.

According to an embodiment, the electronic device 201 may identify that the user's eyes are closed for a predesignated period of time, based on the third signal.

According to an embodiment, the electronic device 201 may output notification information indicating the sleep state of the user, based on a result of the identification.

According to an embodiment, the electronic device 201 may identify the number of the user's heartbeats, based on the third signal.

According to an embodiment, when the heart rate is identified to be greater than the preconfigured number of times, the electronic device 201 may output notification information indicating an abnormality in the number of the heartbeats.

According to an embodiment, the electronic device 201 may identify that the user's jaw moves at a regular interval, based on the third signal.

According to an embodiment, the electronic device 201 may identify whether the user is eating or identify the mealtime of the user, based on a result of the identification.

According to an embodiment, the electronic device 201 may identify that the second movement includes a head movement of the user, an arm movement of the user, or a foot movement of the user.

According to an embodiment, a method for operating an electronic device 201 may include acquiring a third signal for identifying a first movement of the user, from the first wearable electronic device 301 via a communication module of the electronic device 201.

According to an embodiment, in the method for operating the electronic device 201, the third signal may be acquired using a first signal measured by the first wearable electronic device 301 worn in one ear of the user and a second signal measured by a second wearable electronic device 302 worn in the other ear of the user.

According to an embodiment, in the method for operating the electronic device 201, the first signal may include a brainwave signal of the user, a first noise caused by a first movement of the user, and a second noise caused by a second movement of the user, and the second signal may include a third noise caused by the second movement.

According to an embodiment, the method for operating the electronic device 201 may include executing a function of the electronic device corresponding to the first movement, based on the third signal.

According to an embodiment, a non-transitory recording medium may include at least one instruction that enables an operation of acquiring a third signal for identifying a first movement of the user, from the first wearable electronic device 301 via the communication module 290 of the electronic device 201.

According to an embodiment, in the non-transitory recording medium, the third signal may be acquired using a first signal measured by the first wearable electronic device 301 worn in one ear of the user and a second signal measured by a second wearable electronic device 302 worn in the other ear of the user.

According to an embodiment, in the non-transitory recording medium, the first signal may include a brainwave signal of the user, a first noise caused by a first movement of the user, and a second noise caused by a second movement of the user, and the second signal may include a third noise caused by the second movement.

According to an embodiment, the non-transitory recording medium may include at least one instruction that enables an operation of executing a function of the electronic device 201 corresponding to the first movement, based on the third signal.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101, 201), 1st wearable electronic device (301), 2nd wearable electronic device (401). For example, a processor (e.g., the processor 120, 220, 320, 420) of the machine (e.g., the electronic device 101, 201), 1st wearable electronic device (301), 2nd wearable electronic device (401). may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

What is claimed is:

1. A first wearable electronic device comprising:
a first electrode;
a communication circuitry;
a processor; and
memory connected electrically to the processor and configured to store instructions executable by the processor, wherein the instructions, when executed by the processor, cause the first wearable electronic device to:
acquire a first signal via the first electrode in a state where the first wearable electronic device is worn in a first ear of a user, the first signal comprising a brainwave signal of the user, a first noise caused by a first movement of the user, and a second noise caused by a second movement of the user;
acquire, via the communication circuitry, a second signal from a second wearable electronic device worn in a second ear of the user, the second signal being measured by the second wearable electronic device and comprising a third noise caused by the second movement of the user;
acquire, using the first signal and the second signal, a third signal for identifying the first movement; and
transmit the third signal to an electronic device via the communication circuitry such that the electronic device performs a function corresponding to the first movement.

2. The first wearable electronic device of claim 1, wherein the instructions are configured to, when executed by the processor, cause the first wearable electronic device to use the second signal as a reference signal for removing, from the first signal, the second noise caused by the second movement.

3. The first wearable electronic device of claim 1, wherein the first signal is measured by the first electrode of the first wearable electronic device, the first electrode being in contact with a hole of the first ear of the user, and
wherein the second signal is measured by a second electrode of the second wearable electronic device, the second electrode being in contact with a concha of the second ear of the user.

4. The first wearable electronic device of claim 1, wherein the instructions are configured to, when executed by the processor, cause the first wearable electronic device to generate the third signal by removing a portion corresponding to the second noise from the first signal by using the second signal.

5. The first wearable electronic device of claim 1, wherein the first movement comprises an eye blink of the user, a jaw movement of the user, or a heartbeat of the user.

6. The first wearable electronic device of claim 1, wherein the second movement comprises a head movement of the user, an arm movement of the user, or a foot movement of the user.

7. The first wearable electronic device of claim 1, further comprising a ground electrode that is in contact with a concha portion of the first ear of the user.

8. An electronic device comprising:
a communication circuitry;
a processor; and
memory storing instructions,
wherein the instructions, when executed by the processor, cause the electronic device to:
acquire a third signal for identifying a first movement of a user, wherein the third signal is acquired using a first signal measured by a first wearable electronic device worn in a first ear of the user and a second signal measured by a second wearable electronic device worn in a second ear of the user, the first signal comprises a brainwave signal of the user, a first noise caused by the first movement of the user, and a second noise caused by a second movement of the user, and the second signal comprises a third noise caused by the second movement; and
based on the third signal, perform a function of the electronic device, corresponding to the first movement.

9. The electronic device of claim 8, wherein the function of the electronic device comprises a function of controlling a music application of the electronic device, a function of locking or unlocking the electronic device, a function of sensing or outputting information on a sleep state of the user, and a function of sensing or outputting information on an exercise state of the user.

10. The electronic device of claim 8, wherein the instructions, when executed by the processor, cause the electronic device to input the third signal into an AI model prestored in the electronic device to thereby determine whether to perform the function of the electronic device.

11. The electronic device of claim 8, wherein the instructions, when executed by the processor, cause the electronic device to:
input the third signal into an AI model prestored in the electronic device to thereby identify whether the first movement indicated by the third signal corresponds to a predesignated movement related to the function; and
in case that it is identified that the first movement corresponds to the predesignated movement, perform the function of the electronic device.

12. The electronic device of claim 8, wherein the instructions, when executed by the processor, cause the electronic device to:
identify that the user's eyes are closed for a preconfigured period of time, based on the third signal; and
based on a result of the identification, output notification information indicating that the user is in a sleep state.

13. The electronic device of claim 8, wherein the instructions, when executed by the processor, cause the electronic device to:
identify the user's heart rate, based on the third signal; and
in case that the user's heart rate is identified to be greater than a preconfigured number of times, output notification information indicating an abnormality in the user's heart rate.

14. The electronic device of claim 8, wherein the instructions, when executed by the processor, cause the electronic device to:
identify that the user's jaw moves at a regular interval, based on the third signal; and
based on a result of the identification, identify whether the user is eating or identify a mealtime of the user.

15. The electronic device of claim 8, wherein the instructions, when executed by the processor, cause the electronic device to:
acquire the first signal and the second signal from the first wearable electronic device via the communication circuitry; and
acquire the third signal by using the second signal as a reference signal for removing, from the first signal, the second noise caused by the second movement.

16. A method for operating an electronic device, the method comprising:
acquiring first and second signals or a third signal for identifying a first movement of a user, wherein the third signal is acquired using a first signal measured by a first wearable electronic device worn in a first ear of the user and a second signal measured by a second wearable electronic device worn in a second ear of the user, the first signal comprises a brainwave signal of the user, a first noise caused by the first movement of the user, and a second noise caused by a second movement of the user, and the second signal comprises a third noise caused by the second movement; and
based on the third signal, performing a function of the electronic device, corresponding to the first movement.

17. The method of claim 16, further comprising:
inputting the third signal into an AI model prestored in the electronic device to thereby determine whether to perform the function of the electronic device.

18. The method of claim 16, wherein performing the function of the electronic device comprises:
identifying the user's heart rate, based on the third signal; and
in case that the user's heart rate is identified to be greater than a preconfigured number of times, outputting notification information indicating an abnormality in the user's heart rate.

19. The method of claim 16, wherein acquiring the third signal comprises:
acquiring the first signal and the second signal from the first wearable electronic device via communication circuitry of the electronic device; and
acquiring the third signal by using the second signal as a reference signal for removing, from the first signal, the second noise caused by the second movement.
